(19) United States
(12) Patent Application Publication
(10) Pub. No.: US 2009/0311240 A1
(43) Pub. Date: Dec. 17, 2009

(54) VEGF165 Delivered by Fibrin Sealant to Reduce Tissue Necrosis

(75) Inventors: Rainer Mittermayr, Vienna (AT); Sam L. Helgerson, Lincolnshire, IL (US); Heinz Redl, Vienna (AT)

Correspondence Address: MCANDREWS HELD & MALLOY, LTD, 500 WEST MADISON STREET, SUITE 3400, CHICAGO, IL, 60661, US

(73) Assignees: Baxter International Inc., Deerfield, IL (US); Baxter Healthcare S.A., Wallisellen (CH)

(21) Appl. No.: 12/470,816

(22) Filed: May 22, 2009

Related U.S. Application Data
(60) Provisional application No. 61/128,694, filed on May 22, 2008.

(51) Int. Cl. A61K 38/18; A61K 38/48

(52) U.S. Cl. 424/94.64; 514/12

(57) ABSTRACT

The present application demonstrates the clinical potential of fibrin sealants to locally deliver growth factors to ischemic tissue. More particularly, it demonstrates that hydrogels such as Fibrin Sealants can be used to deliver VEGF₁₆₅ to prevent tissue necrosis caused by hypoxia or ischemia. Specifically, a Fibrin Sealant (FS) was used to deliver VEGF₁₆₅ to treat tissue necrosis in both a rodent dorsal flap model and a rodent epigastric flap model. Flaps treated with FS spiked with (rh)VEGF₁₆₅ developed less necrotic tissue. In addition, immunohistological studies revealed a greater numbers of blood vessels (angiogenesis).

### RELATED APPLICATIONS

The present application is a nonprovisional of U.S. Patent Application No. 61/128,694, which was filed May 22, 2008. The entire text of the aforementioned application is incorporated herein by reference in its entirety.

### FEDERALLY SPONSORED RESEARCH OR DEVELOPMENT

[Not Applicable]

### BACKGROUND OF THE INVENTION

Tissue ischemia is a serious complication in numerous surgical specialties often leading to extensive surgical revisions, especially in patients suffering from diabetic/peripheral pathologies (e.g. atherosclerosis, disturbed/delayed wound healing). Insufficient arterial (in-)flow with the accompanying decreased nutritional supply to hypoxic/ischemic tissues can potentially be overcome by therapeutic angiogenesis (Hockel et al., Arch Surg, 128:423-429 (1993)). Numerous angiogenic factors have been studied for efficacy (Pepper, Arterioscler Thromb Vasc Biol, 17:605-619 (1997); Vranckx et al., Wound Repair Regen, 13:51-60 (2005); Zhang et al., Microsurgery, 24:162-167 (2004)). The administration mode of angiogenic factors have also been studied, as the intended clinical use and efficacy are a concern when known adverse effects occur with systemic administration (Eppler et al., Clin Pharmacol Ther, 72:20-32 (2002); Kryger et al., Br J Plast Surg, 53:234-239 (2000); Yang et al., J Pharmacol Exp Ther, 284:103-110 (1998)).

The vascular endothelial growth factor (VEGF) exhibits a potent ability to induce de novo formation of vessels from preexisting vascular structures. In vivo, VEGF is an endogenous inducer of both increased permeability of blood vessels and angiogenesis, thus playing a crucial role in the regulation of neo-vascularization in tissues.

Partial beneficial effects of VEGF in reducing flap necrosis was demonstrated for various administration routes (transvascular, intradermal, subfascial) using either the recombinant protein in liquid formulation or by gene delivery techniques (Kryger et al., Br J Plast Surg, 53:234-239 (2000)).

However, since the VEGF protein has a short half-life in vivo other approaches have been used, e.g. liposome (Liu et al., Wound Repair Regen, 12:80-85 (2004)) or virally mediated gene delivery (Deodato et al., Gene Ther, 9:777-785 (2002); Vranckx et al., Wound Repair Regen, 13:51-60 (2005)), to enhance the long term protein expression. The efficacy of such approaches was also shown to be beneficial in experimental flap survival (Giunta et al., J Gene Med, 7:297-306 (2005); Yang et al., Br J Plast Surg, 58:339-347 (2005)). However, there are serious concerns about the safety of viral gene therapy (Felgner et al., Nature, 349:351-352 (1991)). Thus the sustained delivery of VEGF₁₆₅ by non viral means would offer significant advantages over these practices.

Fibrin Sealants (FS) can be used as a biodegradable biomatrix for local delivery of bioactive substances due to its open porous microstructure and its capacity to reversibly bind specific growth factors (Helgerson et al., Fibrin. N.Y.: Marcel Dekker, Inc., 2004, pp 603-610). For example, a study showed that fibrinogen has specific binding sites for VEGF₁₆₅ (Sahni et al., Blood, 96:3772-3778 (2000)).

Studies of angiogenesis showed that growth factors can be effectively delivered from FS (Arkudas et al., Mol Med, 13:480-487 (2007); Pandit et al., Growth Factors, 15:113-123 (1998); Wong et al., Thromb Haemost, 89:573-582 (2003)). Furthermore, it was shown that FS itself has pro-angiogenic effects (Wong et al., Thromb Haemost, 89:573-582 (2003)).

### BRIEF SUMMARY OF THE INVENTION

Localized and prolonged release of growth factors via FS for sustained pro-angiogenic stimulus and focused delivery to ischemic tissue would be clinically favorable. Thus, the present studies use FS as a sprayed delivery biomatrix for naturally bound growth factors to reduce tissue necrosis, thus resulting in an enhanced survival of tissue flaps. Thereby, (rh)VEGF₁₆₅ bound to FS is locally administered to the recipient flap site.

Accordingly, the present invention relates to a method of increasing neovascularization at the site of a tissue implant comprising applying locally to said site a fibrin sealant composition comprising a growth factor that induces angiogenesis. In such methods the tissue implant has an increased survival rate as compared to a tissue implant that is not treated with a fibrin sealant composition comprising a growth factor. Preferably, the tissue implant exhibits less shrinkage than a comparable tissue implant that has not been treated with a fibrin sealant composition comprising a growth factor.

Another advantageous use of the invention relates to reducing tissue necrosis at a wound site comprising contacting said wound site with a fibrin sealant composition comprising a growth factor that induces angiogenesis, wherein tissue necrosis at the wound site is decreased in the presence of said fibrin sealant as compared to administration of said growth factor alone.

Also contemplated is a method of enhancing wound repair comprising contacting said wound site with a fibrin sealant composition comprising a growth factor that induces angiogenesis.

The methods of the invention also may be used to decrease tissue ischemia at a wound site or site of tissue graft comprising contacting said wound site or tissue graft with a fibrin sealant composition comprising a growth factor that induces angiogenesis, wherein tissue necrosis at the wound site is decreased in the presence of said fibrin sealant as compared to administration of said growth factor alone.

In the methods contemplated herein the growth factor that induces angiogenesis is a vascular endothelial growth factor (VEGF). Preferably, the VEGF is a recombinant human VEGF (rhVEGF). In specific embodiments, the VEGF is preferably selected from the group consisting of VEGF₁₂₁, VEGF₁₄₅, VEGF₁₆₅, VEGF₁₈₃, VEGF₁₈₉, VEGF₂₀₆, VEGF-B, VEGF-C, VEGF-D, VEGF-E, placental growth factor (PIGF) and endocrine gland-derived VEGF (EG-VEGF). In particularly preferred embodiments, the rhVEGF is rhVEGF₁₆₅.

The fibrin sealant used herein may be any fibrin sealant. Typically, the sealant is a sealant that comprises: a sealer protein component; and a thrombin component reconstituted in CaCl₂; wherein said fibrin sealant comprises said VEGF in either the sealer protein component or in the thrombin component. Optionally, in some embodiments, the composition may further comprise a fibrinolysis inhibitor. Exemplary commercially available sealants include TISSEEL VH™ and ARTISS™. Preferably, the sealant is configured as a sealant spray.

In the sealants, the thrombin component in said sealant is between 0.5 IU to about 1000 IU/ml thrombin rendering the sealant as a sprayable format.

Also described is a kit for use in wound healing or tissue repair, said kit comprising: a tissue sealant sealer protein component; a fibrinolysis inhibitor component; a thrombin component reconstituted in CaCl₂ and a VEGF component.

Also contemplated is a composition for healing skin grafts comprising thrombin, fibrinogen, a fibrinolysis inhibitor and VEGF₁₆₅. Preferably the composition is reconstituted immediately prior to application as a liquid or spray. The concentration of fibrinogen in the composition is preferably between 10 and 250 mg/ml. The concentration of thrombin in the composition is preferably between about 1.0 U/ml and about 2.5 U/ml. Alternatively, the thrombin component may be orders of magnitude greater than this amount and may for example be 500 IU. It is contemplated that the composition may include 2, 4, 6, 8, 10, 15, 20, 25, 30, 35, 40, 45, 50 IU thrombin or multiples of this figure such as e.g., 100 IU, 150 IU, 200 IU, 250 IU, 300 IU, 350 IU, 400 IU, 450 IU, 500 IU, 550 IU, 600 IU, 650 IU, 700 IU, 750 IU, 800 IU, 850 IU, 900 IU, 950 IU, or 1000 IU thrombin. The concentration of thrombin is preferably about 4 IU/ml or 5001 U/ml and the concentration of fibrinogen is preferably between about 75 and about 150 mg/ml. In specific embodiments, the fibrinolysis inhibitor is aprotinin, which may be present at between about 1,000 KIU/ml and about 10,000 KIU/ml. In certain embodiments, the aprotinin is about 3000 KIU/ml.

The composition may further comprise one or more molecules selected from the group consisting of a polypeptide growth factor, a cytokine, an enzyme, a hormone, an antibiotic, a protease inhibitor, and an antimycotic, or a combination thereof.

The invention also describes a method of treating a wound comprising preparing a fibrin sealant, comprising: a) mixing equivalent volumes of a first solution comprising fibrinogen and a second solution comprising thrombin reconstituted in a CaCl2 solution, wherein the concentration of thrombin is preferably about 4 IU/ml or 500 IU/ml or an integer therebetween; and wherein said first solution and/or said solution further comprise VEGF165 b) distributing said mixture onto a wound surface, such that a fibrin sealant is formed on said wound.

### BRIEF DESCRIPTION OF SEVERAL VIEWS OF THE DRAWINGS

FIG. 1: Planimetric analysis. (A) Viable flap area (% of total flap area) assessed by planimetry over a 14-day interval. The recombinant human vascular endothelial growth factor (rhVEGF₁₆₅)-treated group showed extended areas of viable flap tissue relative to other groups at all time points, with a significant difference on day 14 relative to control (p<0.01). (B) Reduced flap necrosis was seen between the control group and the FS/VEGF group over the entire observation period, with statistical significance on day 14 (p<0.05). (C) Flap shrinkage at day 14 was less pronounced in the FS/VEGF group, but not significantly different relative to controls. Data are presented as mean_standard error of mean. n.s., not significant; FS, fibrin sealant; VEGF, vascular endothelial growth factor.

FIG. 2: Evaluation of induced angiogenesis. Von Willebrand factor, a marker for endothelial structures, positively stained vessels were counted in the proximal, middle, and distal thirds of the skin flap at 200× magnification. Means were calculated from each slide and an average vessel density was calculated per sample. Data are presented as mean_standard error of mean. * Differs significantly (p<0.05) from control.

FIG. 3: Percentage of flap necrosis on day 3 and day 7 after surgery. The FS group with VEGF₁₆₅ at 200 ng and 400 ng/mL final FS clot showed statistically significant superior effectiveness in reducing necrosis on day 3 as well as on day 7 post surgery compared to control. Fibrin sealant by itself had also reduced areas of necrotic tissue than compared to control, although not statistically significant. The lowest VEGF₁₆₅ concentration (20 ng/mL final FS clot) showed no additional effect in reducing necrosis in comparison to fibrin sealant alone. In the control group, tissue necrosis became also more pronounced (˜25% of entire flap) during the day 3-7 interval. Data are presented as means±SEM. * FS +400 ng VEGF₁₆₅/mL final FS clot (p<0.05); # FS +200 ng VEGF₁₆₅/mL final FS clot (p<0.05).

FIG. 4: Flap perfusion assessed in relative perfusion units (PU) with the Laser Doppler Imaging (LDI) System. Percent deviation from baseline value (=preOP=100%) are given. Ligation and dissection of one of the inferior epigastric neurovascular bundles (left or right according to randomization protocol) resulted in a substantial decrease in flap perfusion to approx. 40% of pre-surgical values in all groups without any differences between the groups indicating same baseline conditions for all groups.

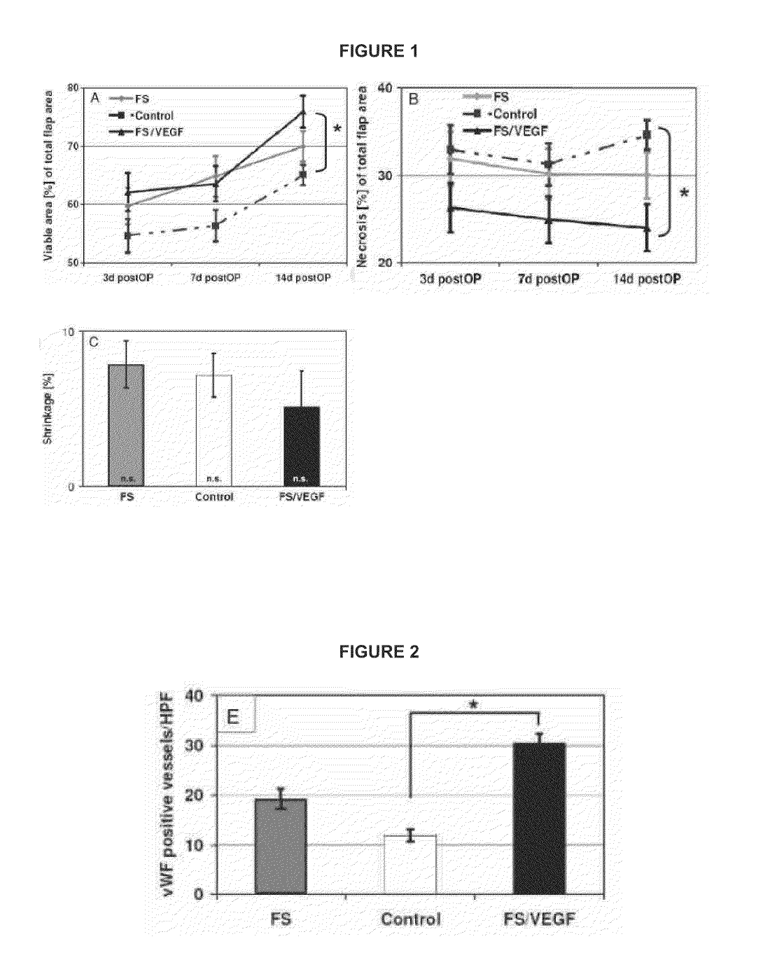

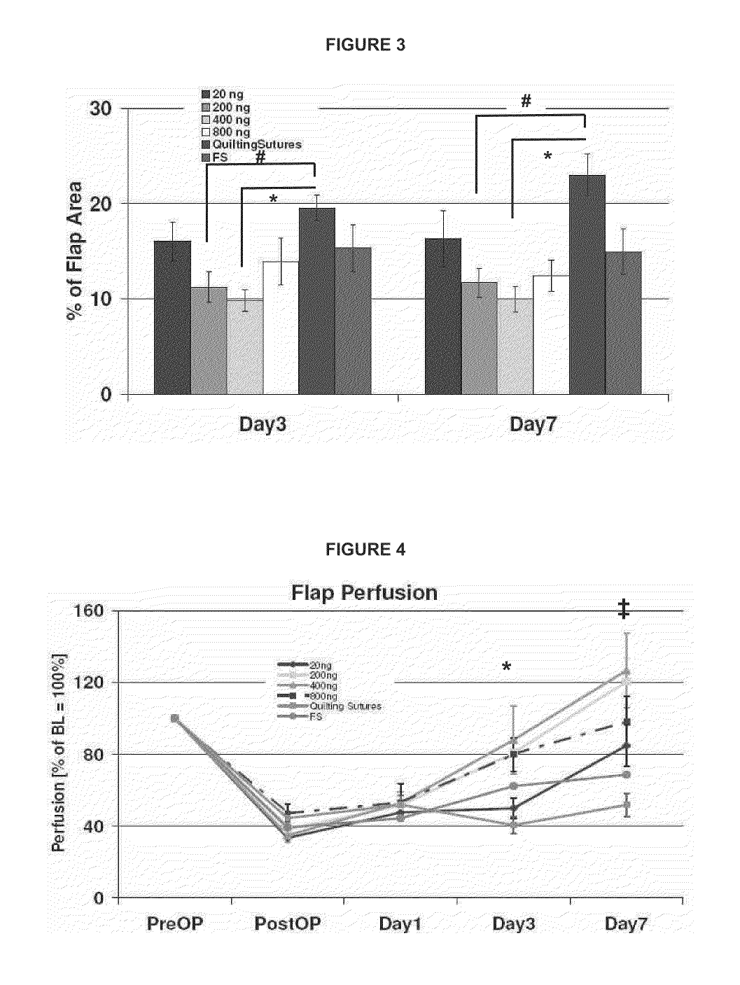

Already on the 3^{rd} postoperative day FS groups having 200, 400, and 800 ng VEGF₁₆₅ incorporated showed a statistically significant improvement in perfusion in comparison to the control group (* 200, 400, 800 ng VEGF165 vs. control—p<0.05). On day 7 post postoperatively a further improvement in perfusion was determined in all FS groups. However, only FS groups with 200 and 400 ng VEGF₁₆₅ had a significantly higher perfusion in comparison to the control group (‡200 and 400 ng VEGF165 vs. control p<0.05). The control group showed only approximately 50% flap perfusion of baseline on day 7. Data are presented as means±SEM.

### DETAILED DESCRIPTION OF THE INVENTION

As noted above, tissue ischemia provides serious complications in wound healing and there is a need to provide an effective method of therapeutic angiogenesis in such wound healing applications. The invention demonstrates the clinical potential of fibrin sealants to locally deliver growth factors to ischemic tissue. Methods and compositions for achieving therapeutic outcome using these findings are described herein.

In particular fibrin sealant compositions are prepared and will be used as local delivery devices for the administration of VEGF and other angiogenic factors such that the ischemia in grafts, tissue implants or wound sites is reduced. The fibrin sealant compositions are formed by the coagulation of plasma proteins including fibrinogen in the presence of thrombin. This coagulation is chiefly the result of the formation of a polymerized fibrin network, which imitates the formation of a blood clot. In such sealants, thrombin converts fibrinogen to fibrin by enzymatic cleavage, and also converts protransglutaminase (factor XIII) to an active transglutaminase (factor XIIIa). Calcium accelerates the proteolytic activity of thrombin and is a cofactor of FXIII. To form a fibrin sealant for use in the present invention, coagulation is carried out under conditions that are conducive to the formation of a sprayable film.

The fibrin sealant is prepared by combining a solution of a plasma protein such as fibrinogen with a solution of thrombin that is prepared in the presence of CaCl2 such that a fibrin matrix forms. It is contemplated that either the fibrinogen solution or the thrombin solution or both contain VEGF. The thrombin solution includes a solution containing thrombin and any concentration of calcium. However, it should be understood that in some aspects the fibrin sealant may be produced by contacting fibrinogen with thrombin even in the absence of calcium as the calcium merely accelerates the proteolytic activity of thrombin.

The fibrinogen used in the tissue sealants described herein may be obtained from human plasma, (e.g., obtained from blood donors) or can be recombinant fibronogen. The fibrinogen may be either in a liquid form or in the freeze-dried or lyophilized form. If in solid form (such as freeze-dried or lyophilized), the fibrinogen must be reconstituted, e.g., in an isotonic solution. Typically, such an isotonic solution is isotonic sodium chloride containing calcium chloride. The concentration of sodium chloride may be in the range of about 0.5% to about 5.0%, preferably in the range of about 1.0% to about 3.0%, and the concentration of calcium chloride may be in the range of about 0.5 mM to about 50 mM, preferably in the range of about 1 mM to about 10 mM. In other preferred embodiments the calcium chloride is present in about 40 mM, which is in excess of the calcium chloride concentration that is needed for the tissue sealant. The isotonic solution may further comprise one or more protease inhibitors, e.g., a polyvalent protease inhibitor such as aprotinin, provided in a concentration range of about 1,000-10,000 KIU/ml (kallikrein inhibitor units/ml), preferably about 3000 KIU/ml. Alternatively, such protease inhibitor(s) in solution may be added directly to the fibrinogen to reconstitute the protein. The concentration of fibrinogen is usually about 1-1000 mg/ml, preferably 10-250 mg/ml, more preferably 50-150 mg/ml, and most preferably 70-110 mg/ml, however, in certain applications (e.g with embedded cells) diluted formulations might be used such as 60 mg/ml or less. The fibrinogen solution may further contain other plasma proteins such as for example, fibronectin, Factor VIII and Factor XIII.

The thrombin may also be derived from natural sources or may be recombinant or synthetic. If in solid form, thrombin preferably although not necessarily, is reconstituted in an isotonic solution containing calcium, e.g., 1.1% NaCl containing 1 mM calcium chloride. The concentration of the thrombin solution is usually about 0.1-10 U/ml, preferably 0.5-5.0 U/ml, even more preferably 1-3 U/ml and most preferably 2.5 U/ml. Units of thrombin refer to the activity standard as defined by the NIH standard. One NIH unit corresponds to 1.15 International Units. (See, e.g., Gaffney et al. (1995) J. Thromb. Haemost. 74:900-3). Thrombin may also be combined with fibrinogen in the absence of calcium. However, those skilled in the art will recognize that the presence of calcium accelerates the proteolytic activity of thrombin, thus the presence of calcium chloride in the thrombin component of the fibrin sealant is preferred.

In the present invention, the VEGF165 is added to either the fibrinogen solution or to the thrombin solution. VEGF is the most potent and ubiquitous vascular growth factor known and will be the preferred growth factor used to induce a beneficial reduction in ischemia in tissue at a wound site and/or skin graft site. VEGF is also known as VEGF-A, and was the first member of the VEGF family of structurally related dimeric glycoproteins belonging to the platelet-derived growth factor superfamily to be identified. Beside the founding member, the VEGF family includes VEGF-B, VEGF-C, VEGF-D, VEGF-E, placental growth factor (PIGF) and endocrine gland-derived VEGF (EG-VEGF). Active forms of VEGF are synthesised either as homodimers or heterodimers with other VEGF family members. VEGF-A exists in six isoforms generated by alternative splicing; VEGF₁₂₁, VEGF₁₄₅, VEGF₁₆₅, VEGF₁₈₃, VEGF₁₈₉ and VEGF₂₀₆. These isoforms differ primarily in their bioavailability, with VEGF₁₆₅ being the predominant isoform (Podar, et al. 2005 Blood 105(4):1383-1395). While the examples presented herein show the beneficial effects of VEGF165 when delivered to a wound site in a fibrin sealant, it is contemplated that the fibrin sealant may comprise one or more of VEGF₁₂₁, VEGF₁₄₅, VEGF₁₆₅, VEGF₁₈₃, VEGF₁₈₉, VEGF₂₀₆, VEGF-B, VEGF-C, VEGF-D, VEGF-E, placental growth factor (PIGF) and endocrine gland-derived VEGF (EG-VEGF). Other growth factors that been shown to be involved in the regulation of angiogenesis include fibroblast growth factors (FGFs), platelet-derived growth factor (PDGF), transforming growth factor .alpha. (TGF.alpha.), and hepatocyte growth factor (HGF). See, for example, Folkman et al., “Angiogenesis”, J. Biol. Chem., 1992 267 10931-10934 for a review. Any of these factors also may be employed either alone or in combination with VEGF₁₆₅ in the fibrin sealants described herein.

The fibrinogen solution and the thrombin solution are combined (usually in equal volumes) preferably immediately before application to the wound before clotting occurs. Once clotting occurs, a fibrin sealant containing the VEGF165 is formed. Alternatively, the two solutions may be sprayed directly onto the wound site simultaneously using two syringes interconnected by a mixing coupling. Generally, the fibrin sealant formed by the combination of the thrombin and the fibrinogen solutions will be transparent. The volume of the solution containing fibrinogen and thrombin used is dependent upon the thickness of the fibrin sealant desired. Typically, about 2.5 ml of each solution is used for approximately every 100 cm² of surface.

While the above description provides a teaching of the preparation of fibrin sealants from individual component parts, be those components from a natural source (e.g., plasma of an animal) or recombinantly produced, the present invention may advantageously use commercially available fibrin sealants. Exemplary fibrin sealants that can be used in the methods of the present invention include e.g., ARTISS™, TISSEEL™, TISSEEL VH™, Crosseal™, CoStasiS™, Evicel™, BIOSTAT BIOLOGX™, CryoSeal® Fibrin Sealant, Hemaseel(R)HMN™ and the like.

The VEGF-containing fibrin sealant according to the invention also may be seeded with cells for cell culture, particularly keratinocyte cultures, such as human keratinocyte cultures. These cell cultures can be either primary cultures derived from skin biopsies obtained from a patient that have undergone between 1 and 6 or more passages in 1/15 to 1/20 dilutions, or cells preserved in the form of banks in liquid nitrogen. Cells may be cultured in the presence of a feeder cell layer, such as a layer of lethally-irradiated human fibroblasts (See Limat et al., 1986 J Invest Dermatol. October 1986;87(4):485-8). Such cells may be grown to confluence or even sub-confluent density, trypsinized, suspended in an appropriate culture medium, and added to the fibrin sealant immediately upon formation of the fibrin sealant or alternatively, the cells may be added to the mixture of thrombin and fibrinogen prior to coagulation, such that the cells are embedded within the fibrin sealant.

The use of the fibrin sealant containing VEGF165 or other angiogenic agent can be adapted in multiple ways. For example, according to one method of use, the fibrin sealant is prepared in the form of a film, by mixing its two constituents (thrombin, calcic thrombin and fibrinogen) in a culture dish. The layer of fibrin sealant can then be placed on the wound as a complete layer with or without a temporary support such as gauze. Advantageously, this layer of fibrin sealant may also be seeded with cells or other materials that support the healing of the wound or skin graft.

According to another method of using the fibrin sealant of the invention, the two constituents of the support are mixed with in such a way as to integrate the VEGF165 and any other materials to be applied to the wound site within the sealant matrix that is subsequently formed. This method may also be carried out directly on a wound site on a patient, which has been prepared to receive a graft, by spraying a mixture of the fibrin sealant containing the VEGF165 onto the wound using a vector gas (nitrogen) at a pressure of 1 to 2.5 bars, or by applying a paste of the fibrin sealant to the wound.

According to a further method of using the fibrin sealant according to the invention, the two constituents of the support are mixed to form a viscous foam to adhere to a wound. Preferably, the resulting paste is both biodegradable and biocompatible. The paste may be applied to the wound as needed, for example, once weekly. Application of the cell paste according to this embodiment facilitates the induction of granulation tissue and the stimulation of wound closure.

In certain embodiments of the invention, the support further contains one or more disinfectants, preferably methylene blue, and/or one or more drugs selected from antibiotics, and biological response modifiers such as cytokines and wound repair promoters. Preferably, these compounds are included in an amount up to 1% by weight in terms of the total dry weight of fibrin plus thrombin. As used herein, the term “biological response modifiers” refers to substances that are involved in modifying a biological response, such as wound repair, in a manner which enhances a desired therapeutic effect of the fibrin sealant. Examples of suitable biological response modifiers include cytokines, growth factors, wound repair promoters, and the like.

In some examples, it may be useful to include cells within the VEGF165 containing fibrin sealants.

The sealants may be applied by spraying. The spraying can be carried out using a vector gas (e.g., nitrogen at a pressure of 1 to 2.5 bars) or any other method known to those skilled in the art. This spraying does not damage the sealant or denature the polypeptides and when the mixture is sprayed, in a very thin layer is formed directly onto a wound.

### EXAMPLES

### Example 1

In this study we evaluated the efficacy of FS spiked with VEGF₁₆₅ to stimulate blood vessel growth and to reduce tissue necrosis in a dorsal flap rat model.

Thirty healthy Sprague Dawley rats (n=10/group), weighing between 350 and 450 g, were caged individually in stainless steel cages in open housing conditions at a mean room temperature of 18±2° C. with water ad libitum and free dietary access. Each rat was box-induced using Isoflurane and maintained under anesthesia using ketamine (60 mg/kg, IM) and xylazine (16 mg/kg, IM). Fluid substitution was performed by subcutaneous injection of Ringer's solution (1 ml/hour). Following induction of general anesthesia, the back of each animal was shaved and depilated. The animal's rectal temperature was measured and maintained between 36.0 and 38.0° C. throughout the surgery. A rectangular dorsal myocutaneous flap (approximately 10×3 cm²) was harvested from cranially to caudally by blunt dissection and remained attached along the caudal edge. The surgical borders were chosen according to anatomical landmarks (cranially, caudal scapular angle; caudally, christae spinae illiacae). Following flap elevation, the animals were assigned randomly to one of three groups.

Animals in the control group were simply sutured to original anatomical and orientation and were not further treated. Animals in the FS group were treated with sprayed FS. Animals in the FS/VEGF group were treated with sprayed FS spiked with (rh)VEGF₁₆₅. FS and FS/VEGF were prepared as follows: The 2.0 ml two-component FS Tisseel VH® (Baxter AG, Austria) was used in this study; The Sealer Protein component (Fibrinogen 75-115 mg/ml) was reconstituted with fibrinolysis inhibitor solution (Aprotinin 3,000 KIU/ml) and spiked with (rh)VEGF₁₆₅ (200 ng/ml); The Thrombin component (500 IU/ml) was reconstituted with CaCl₂ (40 μmol/ml) and diluted to 4 IU/ml.

The fibrin sealant hydrogel was applied to the recipient bed using a spray device (Tissomat™, Baxter AG) at 0.05 ml/cm². Following therapeutic intervention, the flap was immediately repositioned to the original anatomic orientation. Concomitant gentle pressure was applied onto the flaps allowing complete polymerization of the FS. Flaps were sutured to the recipient bed using 4/0 non-resorbable polyester suture in a simple interrupted pattern. Buprenorphine (2.0-2.5 mg/kg, SC) was administered for postoperative analgesia. Animals were anesthetized on day 0, 3, 7 and 14 to perform planimetric analyses. Flap adherence to the recipient bed was macroscopically evaluated on day 14 and scored 1 (no flap adherence) to 3 (complete flap adherence). Animals were euthanized with an overdose of pentobarbital on day 14 and full thickness samples of the entire flap length were taken for standard histological examination and immunohistochemistry.

Tensile Strength

Specimens of flap with adjacent normal tissue were harvested, and immediately fixed to aluminum blocks with cyanoacrylate glue (Indermil, Auto Suture, USA). The tensile strength was then measured using a Universal Materials Testing Machine (Instron™ Type 4301, UK) at a tensile loading speed of 5 mm/min to determine the maximum load (Load at Peak [N]).

Tensile strength was greatest at the junction between flap tissue and adjacent normal tissue for the FS/VEGF treated group (8.5±0.6 N). Tensile strength in the FS group and the Control group had nearly identical tensile strength results (7.5±0.5 N. 7.3±0.4 N; respectively), and were less resistant to dispersion force compared to the FS/VEGF group.

All proximal regions in the FS and FS/VEGF groups had complete adherence. And, all but one middle region in the FS group had complete adherence in the FS group and FS/VEGF group. Only 7 of 10 proximal and 4 of 10 middle regions had complete adherence in the Control group.

This work revealed that (rh)VEGF₁₆₅ increases the number of blood vessels and the viability of tissue flaps in vivo.

The results clearly show clinical improvement of ischemic tissue by the effective release of (rh)VEGF₁₆₅ from sprayed FS matrices.

Clinical efficacy was clearly demonstrated by significantly reducing tissue necrosis concomitant with greater viable flap area and less shrinkage in FS/VEGF treated groups.

### Example 2

In this study we investigated local sprayed fibrin sealant supplemented with VEGF₁₆₅ at various concentrations on tissue necrosis in a rodent epigastric flap model. The efficacy to reduce tissue necrosis was observed over a 1 week period by digital photography and data were evaluated by a planimetric evaluation software tool. Furthermore, the influence of locally delivered VEGF₁₆₅ from FS on superficial flap perfusion was tested using laser Doppler imaging.

This was a prospective, controlled, randomized, pre-clinical study. To test efficacy of FS supplemented with ascending concentrations of VEGF isoforms 165, epigastric fasciomyocutaneous flaps were harvested and treated with local sprayed FS ±VEGF₁₆₅ on the recipient site. Four dosages of VEGF₁₆₅ were tested (=test items): 20 ng/ml final FS clot, 200 ng/ml final FS clot, 400 ng/ml final FS clot, and 800 ng/ml final FS clot. FS without any VEGF₁₆₅ served as the reference item.

Test Items:

A. Sprayed FS (TISSEEL VH S/D, DUO4 Two-Component Fibrin Sealant, deep frozen) at 0.01 ml/cm² with
i. 20 ng VEGF₁₆₅/mL final FS clot
ii. 200 ng VEGF₁₆₅/mL final FS clot
iii. 400 ng VEGF₁₆₅/mL final FS clot
iv. 800 ng VEGF₁₆₅/mL final FS clot

Reference Item

B. Sprayed FS (TISSEEL VH S/D, DUO4 Two-Component Fibrin Sealant, deep frozen) at 0.01 ml/cm²

Control:

C. Quilting Sutures; used to obtain flap adherence to the recipient bed

### Example 3

### (rh)VEGF₁₆₅ Release from an In Vitro Fibrin Matrix

The 2.0 ml two-component FS Tisseel VH® (Baxter AG, Austria) was used in this study. The Sealer Protein component (Fibrinogen 75-115 mg/ml) was reconstituted with fibrinolysis inhibitor solution (Aprotinin 3,000 KIU/ml) and spiked with (200 ng/ml). The Thrombin component (500 IU/ml) was reconstituted with CaCl₂ (40 μmol/ml) and diluted to 4 IU/ml.(18) Five fibrin matrices were made by mixing 75 μl of the Sealer Protein and (rh)VEGF₁₆₅ solution with 75 μl of the thrombin component at 37° C. The resulting 150 μl fibrin matrix contained 200 ng/ml of (rh)VEGF₁₆₅. Each fibrin matrix was individually covered with 1 ml of PBS with Aprotinin at 500 IU/ml and incubated at 37° C. on a shaking plate. At 1, 24, 46, 88 and 97 hours, the supernatant was collected, and fresh PBS and aprotinin was added. At 97 hours, fibrin matrices were lysed with trypsin to determine residual (rh)VEGF₁₆₅ content of the fibrin matrix. (rh)VEGF₁₆₅ was measured using an ELISA (Quantikine® Immunoassay kit, R&D, MN) read at 450 nm.

(rh)VEGF₁₆₅ was rapidly released from the FS matrix starting within the first hour of incubation and was maintained during the first 24 hours (FIG. 2). Release decreased to zero between 24 and 88 hours. The cumulative measured amount of (rh)VEGF₁₆₅ released from the FS clots was approximately 66% of the initial (rh)VEGF₁₆₅ concentration. Lysate of the remaining FS matrix showed no residual VEGF₁₆₅.

### Example 4

### Effects of In Vivo (rh)VEGF₁₆₅ Release on VEGF-R2 Expression

Twenty transgenic FVB/N-Tg(Vegfr2-luc)Xen mice were used. These mice carry a transgene which contains a 4.5-kb murine VEGF-R2 promoter fragment that drives the expression of a firefly lucierase reporter protein. Each mouse was box-induced using isoflurane and maintained under anesthesia with ketamine (60 mg/kg, IP) and xylazine (7.5 mg/kg, IP). Mice were injected with luciferin (150 mg/kg, IP) and imaged with an in vivo imaging system (VivoVision® IVIS®, Xenogen, Calif.) to acquire a background image signal. Each animal's dorsum was then shaved and disinfected. A bilateral subcutaneous tunnel to each flank was bluntly created through a 1 cm incision at the caudal aspect of the neck. Each tunnel was filled or left empty depending on the treatment group. The treatment groups were (1) untreated control to measure endogenous expression of VEFG-R2 due to tunneling, (2) FS group to measure expression of VEFG-R2 due to FS, and (3) FS with (rh)VEGF₁₆₅ (200 ng/ml) to measure the expression of VEGF-R2 due to (rh)VEGF₁₆₅ release. FS implants were fixed with one subcutaneous resorbable suture (Vicryl 5/0, Ethicon, N.J.) to prevent movement. As a fourth group all neck incisions measured the endogenous expression of VEGF-R2 during cutaneous wound healing. A fifth group that did not receive surgery was used to measure the endogenous expression of VEGF-R2 following a (200 μl) intradermal injection of (rh)VEGF₁₆₅ (200 ng/ml) in distilled water. Sealer protein and thrombin solutions were prepared as previously described. FS implants were prepared by mixing 100 μl of Sealer protein with 100 μl of thrombin into a 0.8 cm diameter, 0.4 cm thick disc at 37° C. until fully polymerized. Two implantation sites per animal were randomly assigned to a group for a total of 10 sites per group. Bioluminescence signal was calculated 15 min following an injection of luciferin to measure VEGF-R2 expression. The bioluminescence signal was quantified using Living Image Software (Xenogen) from the in vivo luciferase activity measured in emitted photon counts per second. Pre-surgical activity was set to 100% and the subsequent measurements were referenced to this baseline. Bioluminescence images were collected at 2 hours and on day 1, 2, 5, 7, 10, 13, 16, 19, and 22. Bioluminescence is a signal of VEGF-R2 activity.

The neck incision serving as the access point for bilateral clot implantation to each flank constituted the individual endogenous wound healing response with concomitant VEGF-R2 expression. Associated bioluminescence imaging showed a rapid increase in VEGF-R2 expression in the initial wound healing phase (first 2 days). Thereafter, a continuous decrease in the activity was observed until day 13 (FIG. 5). In comparison, blunt tunneling in the fascial layer showed no impact on the expression activity. The FS clot moderately increased VEGF-R2 expression at day 5. However, addition of (rh)VEGF₁₆₅ to the FS matrix produced a doubling (200% from baseline) of receptor expression in the day 5 to 13 interval and returned to nearly baseline values thereafter. In contrast, intradermal injection of (rh)VEGF₁₆₅ caused only a moderate higher receptor activity relative to endogenous wound healing. In addition, the VEGF intradermal injection showed a more widespread signal around the original application site. This was not observed in the FS matrix implantation groups, where the signal was strongly limited to the immediate vicinity of the implantation site.

In summary, we showed that (rh)VEGF₁₆₅ can be released from a fibrin matrix to stimulate blood vessel growth in vivo and to enhance flap survival subjected to ischemia. Results from the planimetric analysis are consistent with immunohistochemistry which demonstrated enhanced vessel density. This indicates that fibrin sealant contributes to a better flap outcome by its proposed dual mechanism, laminar and maintained flap fixation combined with a sustained local delivery of the growth factor VEGF acting as a potent inducer of angiogenesis. The sustained local delivery of growth factor was confirmed by in vivo up-regulation of VEGF-R2 expression in the transgenic mouse.

1. A method of increasing neovascularization at the site of a tissue implant comprising applying locally to said site a fibrin sealant composition comprising a growth factor that induces angiogenesis.

2. The method of claim 1, wherein said tissue implant has an increased survival rate as compared to a tissue implant that is not treated with a fibrin sealant composition comprising a growth factor.

3. The method of claim 1, wherein said tissue implant exhibits less shrinkage than a comparable tissue implant that has not been treated with a fibrin sealant composition comprising a growth factor.

4. A method of reducing tissue necrosis at a wound site comprising contacting said wound site with a fibrin sealant composition comprising a growth factor that induces angiogenesis, wherein tissue necrosis at the wound site is decreased in the presence of said fibrin sealant as compared to administration of said growth factor alone.

5. A method of enhancing wound repair comprising contacting said wound site with a fibrin sealant composition comprising a growth factor that induces angiogenesis.

6. A method of decreasing tissue ischemia at a wound site or site of tissue graft comprising contacting said wound site or tissue graft with a fibrin sealant composition comprising a growth factor that induces angiogenesis, wherein tissue necrosis at the wound site is decreased in the presence of said fibrin sealant as compared to administration of said growth factor alone.

7. The method of claim 6, wherein said growth factor that induces angiogenesis is a vascular endothelial growth factor (VEGF).

8. The method of claim 7, wherein said VEGF is a recombinant human VEGF (rhVEGF).

9. The method of claim 7, wherein said VEGF is selected from the group consisting of VEGF₁₂₁, VEGF₁₄₅, VEGF₁₆₅, VEGF₁₈₃, VEGF₁₈₉, VEGF₂₀₆, VEGF-B, VEGF-C, VEGF-D, VEGF-E, placental growth factor (PIGF) and endocrine gland-derived VEGF (EG-VEGF).

10. The method of claim 8, wherein said rhVEGF is rhVEGF₁₆₅.

11. The method of claim 6 wherein said fibrin sealant is a sealant that comprises:
a. a sealer protein component;
and
b. a thrombin component reconstituted in CaCl₂;
wherein said fibrin sealant comprises said VEGF in either the sealer protein component or in the thrombin component.

12. The method of claim 11 wherein said method sealant further comprises fibrinolysis inhibitor.

13. The method of claim 11, wherein said fibrin sealant is selected from the group consisting of TISSEEL VH™ and ARTISS™.

14. The method of claim 6, wherein said fibrin sealant is configured as a sealant spray.

15. The method of claim 10 wherein said thrombin component in said sealant is between 0.5 IU to about 1000 IU/ml thrombin.

16. A kit for use in wound healing or tissue repair, said kit comprising:
a. a tissue sealant sealer protein component;
b. optionally containing a fibrinolysis inhibitor component;
c. a thrombin component reconstituted in CaCl₂ and
d. a VEGF component.

17. A composition for healing skin grafts comprising thrombin, fibrinogen, a fibrinolysis inhibitor and VEGF165.

18. The composition of claim 17, wherein the concentration of fibrinogen is between 10 and 250 mg/ml.

19. The composition of claim 17, wherein the concentration of thrombin is between about 1.0 U/ml and about 1000 U/ml.

20. The composition of claim 17, wherein the concentration of thrombin is 4 IU/ml or 500 IU and the concentration of fibrinogen is between about 75 and about 150 mg/ml.

21. The composition of claim 17, wherein said fibrinolysis inhibitor is aprotinin.

22. The composition of claim 21, wherein the concentration of said aprotinin is between about 1,000 KIU/ml and about 10,000 KIU/ml.

23. The composition of claim 21, wherein the concentration of said aprotinin is about 3000 KIU/ml.

24. The composition of claim 17, further comprising one or more molecules selected from the group consisting of a polypeptide growth factor, a cytokine, an enzyme, a hormone, an antibiotic, a protease inhibitor, and an antimycotic, or a combination thereof.

25. A method of treating a wound comprising preparing a fibrin sealant, comprising: a) mixing equivalent volumes of a first solution comprising fibrinogen and a second solution comprising thrombin reconstituted in a CaCl₂ solution, wherein the concentration of thrombin is between about 1.0 U/ml and about 1000 U/ml; and wherein said first solution and/or said solution further comprise VEGF165 b) distributing said mixture onto a wound surface, such that a fibrin sealant is formed on said wound.
